(19) United States
(12) Patent Application Publication
(10) Pub. No.: US 2022/0274835 A1
(43) Pub. Date: Sep. 1, 2022

(54) CARBON NANOTUBE MICROELECTRODES FOR SENSORS, ELECTROCHEMISTRY, AND ENERGY STORAGE

(71) Applicants: University of Cincinnati, Cincinnati, OH (US); A.O. Smith Corporation, Milwaukee, WI (US)

(72) Inventors: Noe Alvarez, Cincinnati, OH (US); Pankaj Gupta, Cincinnati, OH (US); William R. Heineman, Cincinnati, OH (US); Kiera Gazica, Glenshaw, PA (US); Connor Rahm, Goshen, OH (US); Dehua Jiang, Cincinnati, OH (US); Gusphyl Justin, Milwaukee, WI (US); Joshua Smith, Milwaukee, WI (US)

(73) Assignees: University of Cincinnati, Cincinnati, OH (US); A.O. Smith Corporation, Milwaukee, WI (US)

(21) Appl. No.: 17/631,752

(22) PCT Filed: Jul. 31, 2020
(86) PCT No.: PCT/US2020/044389

§ 371 Date: Jan. 31, 2022

Related U.S. Application Data
(60) Provisional application No. 62/882,032, filed on Aug. 2, 2019.
(60) Provisional application No. 62/964,720, filed on Jan. 23, 2020.

(51) Int. Cl. C01B 32/158; A61B 5/263; H01G 11/36; G01N 27/327

(52) U.S. Cl. CPC C01B 32/158; A61B 5/263; H01G 11/36; G01N 27/3278; B82Y 15/00

(57) ABSTRACT

An electrode includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material. A method of analyzing an analyte in a sample and a device for energy storage using the electrode are also described.

### CROSS-REFERENCE TO RELATED APPLICATION

This application claims priority to U.S. Provisional application Ser. No. 62/882,032, filed on Aug. 2, 2019, the entire content of which is incorporated herein by reference. This application further claims priority to U.S. Provisional application Ser. No. 62/964,720, filed on Jan. 23, 2020, the entire content of which is incorporated herein by reference.

### FIELD

The present specification generally relates to carbon nanotube microelectrodes and, more particularly, to carbon nanotube microelectrodes for use in sensors, electrochemistry, and energy storage.

### BACKGROUND

Carbon nanotubes (hereinafter “CNTs”, or in the singular, “CNT”) have the potential to be useful in a wide variety of industrial applications. CNTs exhibit interesting physiochemical properties and structural geometries, as well as nanometer-size dimensions. Further, CNTs have a combination of chemical stability, electrical conductivity, and a large surface area, making CNTs attractive for use in electrodes. However, practical use of CNTs has been limited due to difficulties in assembling CNTs into structures that can be handled and manipulated, difficulties in determining where on the assemblies the CNTs are reactive, and difficulties in attaching metallic components (e.g., wires and cables) to the assemblies.

### SUMMARY

Therefore, a need exists for assemblies incorporating CNTs and methods of making those assemblies that solve the practical limitations enumerated above so that these assemblies may be used in electrodes, sensors, and energy storage devices.

According to one or more embodiments, an electrode includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material.

According to one or more embodiments, a method for analyzing an analyte in a sample includes contacting the sample with a sensor comprising an electrode of the above embodiments, applying an electrical potential to the electrode, and measuring the electrical current in the sample as a result of the applied electrical potential. The sample comprises 100 ppm by weight or less of the analyte.

According to one or more embodiments, a device for energy storage includes a plurality of highly densified carbon nanotube rods. The highly densified carbon nanotube rods includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material.

Additional features and advantages of the embodiments described herein will be set forth in the detailed description which follows, and in part will be readily apparent to those skilled in the art from that description or recognized by practicing the embodiments described herein, including the detailed description which follows, the claims, and the appended drawings.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter. The following detailed description of specific embodiments of the present disclosure can be best understood when read in conjunction with the following drawings, where like structure is indicated with like reference numerals and in which:

Figure (FIG. 1 shows a carbon nanotube rod in accordance with embodiments described herein;

FIG. 2 shows an energy storage device including carbon nanotube rods in accordance with embodiments described herein;

FIG. 3 shows structures of carbon nanotubes during various phases of assembling carbon nanotube rods, in accordance with embodiments described herein: vertically aligned carbon nanotube forest (panel A), individual carbon nanotubes extracted from the vertically aligned carbon nanotube forest (panel B), schematic of the path from a vertically aligned carbon nanotube forest to a carbon nanotube film to a carbon nanotube fiber (panel C), and carbon nanotube fibers of various diameters (panel D);

FIG. 4 shows field emission scanning electron microscopy images of CNT fibers having diameters of 28.22 μm (panel A), 49.14 μm (panel B), and 69.45 μm (panel C), in accordance with embodiments described herein;

FIG. 5 shows an exemplary process of preparing carbon nanotube films in accordance with embodiments described herein;

FIG. 6 shows an exemplary process for attaching an electrical conductive material to a carbon nanotube film in accordance with embodiments described herein;

FIG. 7 shows scanning electron microscopy images of carbon nanotube rods in accordance with embodiments described herein: cross-section of three carbon nanotube rod electrodes embedded within a polymer film at 65× magnification (panel A), cross-section of poorly densified carbon nanotube rod electrodes at 5000× magnification (panel B), cross-section of poorly densified carbon nanotube rod electrodes at 25000× magnification (panel C), cross-section of carbon nanotube rod electrodes at 5000× magnification (panel D), and cross-section of poorly densified carbon nanotube electrodes at 50000× magnification (panel E);

FIG. 8 shows a Raman spectrum of a carbon nanotube rod electrode cross-section in accordance with embodiments described herein;

FIG. 9 shows cyclic voltammograms for a carbon nanotube film composed of a single CNT rod cross-section of 28 μm (panel A), 49 μm (panel B), and 69 μm (panel C), in accordance with embodiments described herein;

FIG. 10 shows cyclic voltammograms for a carbon nanotube film composed of three CNT rod cross-sections of 28 μm (panel A), 49 μm (panel B), and 69 μm (panel C), in accordance with embodiments described herein;

FIG. 11 shows cyclic voltammograms for the oxidation and reduction of the FcMeOH/FcMeOH+ redox couple, recorded at a 10 mV s⁻¹ scan rate, for a carbon nanotube film composed of a single CNT rod cross-section of 28 μm (panel A), 49 μm (panel B), and 69 μm (panel C) in accordance with embodiments described herein;

FIG. 12 shows cyclic voltammograms for the cross section of one (panel A) and three (panel B) non-densified CNT rods recorded over at range of scan rates 5-150 mV·s⁻¹ in accordance with embodiments described herein;

FIG. 13 shows cyclic voltammograms for the oxidation and reduction of K₃[Fe(CN)₆] (panel A) and the FcMeOH (panel B), recorded at a 10 mV s⁻¹ scan rate, in accordance with embodiments described herein;

FIG. 14 shows cyclic voltammograms for the oxidation and reduction of K₃[Fe(CN)₆], recorded at a 10 mV s⁻¹ scan rate, at the sidewall of a carbon nanotube rod, in accordance with embodiments described herein;

FIG. 15 shows cyclic voltammograms for the oxidation and reduction of K₃[Fe(CN)₆], recorded at a 10 mV s⁻¹ scan rate, at the sidewall of a carbon nanotube rod, in accordance with embodiments described herein;

FIG. 16 shows square wave voltammograms for increasing concentrations of dopamine at cross-sections of six identical carbon nanotube rods in accordance with embodiments described herein;

FIG. 17 shows square wave voltammograms for increasing concentrations of serotonin at cross-sections of six identical carbon nanotube rods in accordance with embodiments described herein;

FIG. 18 shows the pH dependence of oxidation potential of dopamine (panel A), serotonin (panel B), epinephrine (panel C), and norepinephrine (panel D) at cross-sections of six identical carbon nanotube rods in accordance with embodiments described herein;

FIG. 19 shows the square wave voltammograms recorded for a mixture of ascorbic acid, dopamine, and uric acid, where the concentration of dopamine was kept constant and ascorbic acid and uric acid concentrations were increased to 500 μM (panel A), and the same for the electrochemical oxidation of 0.5 μM dopamine while increasing the concentration of serotonin up to 10-fold (panel B) at cross-sections of six identical carbon nanotube rods in accordance with embodiments described herein;

FIG. 20 shows the microscopic images of PC12 in culture medium at a time interval of 0 hours (panel A) and 48 hours (panel B).

FIG. 21 shows square wave voltammograms of K⁺ induced dopamine release from the population of PC12 cells and then further spiked standard dopamine solutions of different concentrations at cross-sections of six identical carbon nanotube rods in accordance with embodiments described herein;

FIG. 22 shows anodic stripping voltammograms for increasing concentrations of lead ions in acetate buffer using six identical carbon nanotube rod electrodes in accordance with embodiments described herein;

FIG. 23 shows anodic stripping voltammograms for increasing concentrations of lead ions in drinking water using six identical carbon nanotube rod electrodes with a 300 s deposition time (panel A) and with no deposition time (panel B) in accordance with embodiments described herein; and

FIG. 24 shows anodic stripping voltammograms for increasing concentrations of cadmium ions in drinking water using six identical carbon nanotube rod electrodes with a 300 s deposition time (panel A) and with no deposition time (panel B) in accordance with embodiments described herein.

### DETAILED DESCRIPTION

Electrodes and Sensors Including Carbon Nanotube Rods

Reference will now be made to various embodiments of electrodes, sensors, and energy storage devices incorporating CNTs. In one or more embodiments, an electrode includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material. Various embodiments and properties thereof will be described herein with specific reference to the appended drawings.

Referring to FIG. 1, each of the at least one aligned CNT fiber 10 may have a first end 12 and a second end 14. The first end 12 and the second end 14 may be separated from one another by a body 16.

In embodiments, the at least one aligned CNT fiber 10 may be embedded in the insulating surface layer 18. The insulating surface may be made from epoxy containing resin, solvent- and water-borne polyurethane, polysiloxane, polyphosphazene, synthetic organic polymers that have rigidity for cutting, and mixtures of two more of these. When the at least one aligned CNT fiber 10 is embedded in the insulating surface layer 18, the entire assembly may be referred to as a “carbon nanotube rod” or a “CNT rod.”

As used in this context, “at least one” means that any number of aligned CNT fibers may be embedded in the insulating surface layer. For instance, from 1 to 1000 aligned CNT fibers may be embedded in the insulating surface layer. That is, from 1 to 6, from 1 to 12, from 1 to 24, from 1 to 254, from 1 to 10, from 1 to 9, from 1 to 8, from 1 to 7, from 1 to 6, from 1 to 5, from 1 to 4, from 1 to 3, from 1 to 2, from 2 to 254, from 3 to 254, from 4 to 254, from 5 to 254, from 6 to 254, from 7 to 254, from 8 to 254, from 9 to 254, from 10 to 254, from 2 to 1000, from 3 to 1000, from 4 to 1000, from 5 to 1000, from 6 to 1000, from 7 to 1000, from 8 to 1000, from 9 to 1000, or even from 10 to 1000 aligned CNT fibers may be embedded in the insulating surface layer. It should be understood that the number of aligned CNT fibers embedded in the insulating surface layer may be from any of the lower bounds of such number described herein to any of the upper bounds of such number described herein.

In embodiments, the at least one aligned CNT fiber 10 may be arranged in a generally cylindrical shape, as shown in FIG. 1. However, when multiple CNT fibers 10 are used, the CNT fibers 10 may be arranged as an electrode or microelectrode array or as a single electrode or a single microelectrode. Without intending to be bound by any particular theory, it is believed that the electrode or microelectrode array may provide a larger surface area for performing electrochemistry while maintaining microelectrode physics associated with the mass transport. Such microelectrode characteristics are fast establishment of true diffusional steady-state signal, decreased ohmic drop of potential, and larger signal-to-noise ratio. Further, it is believed that the single electrode or microelectrode may be employed where reduced space or volume is available and microscale features are desirable.

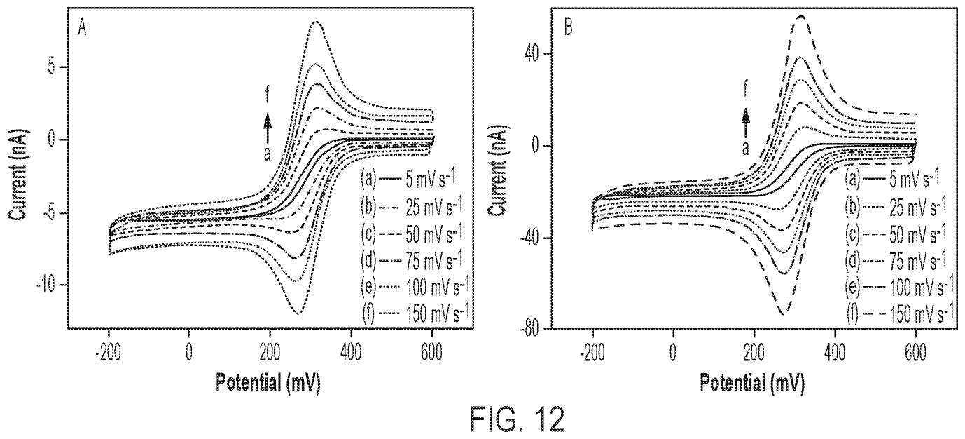

In embodiments, the at least one aligned CNT fiber 10 may be densified. Without intending to be bound by any particular theory, it is believed that densification of the CNT fiber may help to limit the amount of porosity in the CNT fiber 10. As used herein, the term “porosity” refers to the relative amount of open space within the CNT fiber 10, with “high porosity” referring to a large amount of open space within the CNT fiber 10 and “low porosity” referring to a small amount of open space within the CNT fiber 10. Additionally, it is believed that porosity may affect the electrochemical response of the electrodes or sensors formed from the CNT fiber 10. Without intending to be bound by any particular theory, it is believed that porosity will be evident for fibers densified for 30 minutes in acetone (i.e., partial densification), allowing electrolyte migration leading to thin film behavior. These fibers typically lead to transition directly from radial diffusion to thin layer effect as the scan rate increases. In porous CNT fiber, peak to peak separation (ΔE) also decreases (as shown in FIG. 12 described in more detail below, where the ΔE value observed is 42 mV for single fiber, rather than 59 mV). Densification is also believed to improve alignment of the individual CNTs within the CNT fiber 10, discussed in more detail below, and may also increase the conductivity of the CNT fiber 10.

Densification may be accomplished by exposing the CNT fiber 10 to a non-solvent at a temperature and for a period of time. For instance, the non-solvent may be selected from acetone, a mixture of water and acetone, ethylene glycol, N-methyl-2-pyrrolidone, and a mixture of two or more thereof. Densification may take place, for example, for a time ranging from 18 hours to 54 hours, from 22 hours to 50 hours, from 26 hours to 46 hours, from 30 hours to 42 hours, or even from 34 hours to 38 hours. It should be understood that densification may take place for a time ranging from any lower bound for such time described herein to any upper bound of such time described herein. Further, densification may take place, for example, at a temperature ranging from 0° C. to 100° C., from 5° C. to 95° C., from 10° C. to 90° C., from 15° C. to 85° C., from 20° C. to 80° C., from 25° C. to 75° C., from 30° C. to 70° C., from 35° C. to 65° C., from 40° C. to 60° C., or even from 45° C. to 55° C. It should be understood that densification may take place at a temperature ranging from any lower bound for such temperature described herein to any upper bound of such temperature described herein.

In embodiments, each of the at least one aligned CNT fiber is composed of a plurality of CNTs. It is believed that the total number of CNTs in a single CNT fiber, in embodiments, may be one million or more, such as up to 10²³ CNTs. Of course, the total number of CNTs in a single CNT fiber may vary based on the dimensions of the CNT fiber and the like.

The CNTs in a single CNT fiber may have an average length of from 20 μm to 60 μm, from 21 μm to 59 μm, from 22 μm to 58 μm, from 23 μm to 57 μm, from 24 μm to 56 μm, from 25 μm to 55 μm, from 26 μm to 54 μm, from 27 μm to 53 μm, from 28 μm to 52 μm, from 29 μm to 51 μm, from 30 μm to 50 μm, from 31 μm to 49 μm, from 32 μm to 48 μm, from 33 μm to 47 μm, from 34 μm to 46 μm, from 35 μm to 45 μm, from 36 μm to 44 μm, from 37 μm to 43 μm, from 38 μm to 42 μm, or even from 39 μm to 41 μm. It should be understood that the CNTs may have an average length ranging from any lower bound for such length described herein to any upper bound for such length described herein. Without intending to be bound by any particular theory, it is believed that this length may allow for a continuous electron path from the first end of the CNT fiber to the second end of the CNT fiber, which in turn, may allow for fast electron transfer while the CNT fiber is in operation.

Referring again to FIG. 1, in embodiments, the first end 12 and the second end 14 may be free of the insulating surface layer 18. Without intending to be bound by any particular theory, it is believed that assembling the electrode such that the first end 12 and the second end 14 are free of the insulating surface layer 18 allows for access to the first end 12 and the second end 14 to produce electrodes and sensors. Thus, the first end is available for interaction with the target analyte or electrolyte and will be in contact with the appropriate media (aqueous or non-aqueous). Additionally, the first end is thereby available for further functionalization depending on the intended application of the electrode. The second end can then be in contact with an electric conducting material.

In embodiments, the first end 12 may be modified to include one or more hydrogen atoms, one or more carbon atoms, a chemical functional group, a polymer, a nanoparticle, an enzyme, an aptamer, an antibody, a dopant, or a combination of two or more of these.

In embodiments, when a chemical functional group is present, such a chemical functional group may include carboxylic, hydroxyl, amine, silane, thiol, epoxy, oxygen, or a combination of two or more of these.

In embodiments, when a polymer is present, such a polymer may include a conducting polymer, an ion-exchange polymer, a redox polymer, a silyl-modified polymer, a hydrogel polymer, or a combination of two or more of these.

In embodiments, when a nanoparticle is present, such a nanoparticle may include a gold nanoparticle, a silver nanoparticle, a carbon nanoparticle, a palladium nanoparticle, a copper nanoparticle, a platinum nanoparticle, a nickel nanoparticle, or a combination of two or more of these. As used in this context, a “combination of two or more of these” refers to (1) particles comprising two or more metals, e.g. a gold/palladium particle; (2) a mixture of pure particles, e.g. a mixture of gold particles and palladium particles; and/or a combination of these, e.g. a mixture of gold particles, palladium particles, and gold/palladium particles.

In embodiments, when an enzyme is present, such an enzyme may include horseradish peroxidase, glucose oxidase, nicotinamide adenine dinucleotide, organophosphorus hydrolase, cholesterol oxidase, alkaline phosphatase, or a combination of two or more of these.

In embodiments, when an aptamer is present, the aptamer may comprise either chains of oligonucleotides or chains of peptides. For instance, the aptamer may comprise from 20 oligonucleotide to 60 oligonucleotides, from 20 oligonucleotide to 55 oligonucleotides, from 20 oligonucleotide to 50 oligonucleotides, from 20 oligonucleotide to 45 oligonucleotides, from 20 oligonucleotide to 40 oligonucleotides, from 20 oligonucleotide to 35 oligonucleotides, from 20 oligonucleotide to 30 oligonucleotides, from 25 oligonucleotide to 60 oligonucleotides, from 30 oligonucleotide to 60 oligonucleotides, from 35 oligonucleotide to 60 oligonucleotides, from 40 oligonucleotide to 60 oligonucleotides, from 45 oligonucleotide to 60 oligonucleotides, or even from 50 oligonucleotide to 60 oligonucleotides. It should be understood that the aptamer may comprise a number of oligonucleotides ranging from any lower bound for such number described herein to any upper bound for such number described herein. For an aptamer comprising peptides, the aptamer may comprise from 1 to 20 peptides, from 1 to 19 peptides, from 1 to 18 peptides, from 1 to 17 peptides, from 1 to 16 peptides, from 1 to 15 peptides, from 1 to 14 peptides, from 1 to 13 peptides, from 1 to 12 peptides, from 1 to 11 peptides, from 1 to 10 peptides, from 1 to 9 peptides, from 1 to 8 peptides, from 1 to 7 peptides, from 1 to 6 peptides, from 1 to 5 peptides, from 1 to 4 peptides, from 1 to 3 peptides, from 1 to 2 peptides, from 2 to 20 peptides, from 3 to 20 peptides, from 4 to 20 peptides from 5 to 20 peptides, from 6 to 20 peptides, from 7 to 20 peptides, from 8 to 20 peptides, from 9 to 20 peptides, from 10 to 20 peptides, from 11 to 20 peptides, from 12 to 20 peptides, from 13 to 20 peptides, from 14 to 20 peptides, from 15 to 20 peptides, from 16 to 20 peptides, from 17 to 20 peptides, from 18 to 20 peptides, from 19 to 20 peptides.

In embodiments, when an antibody is present, the antibody may be specific to any antigen. Antigens may originate from any pathogen, including pathogenic bacteria and viruses.

In embodiments, when a dopant is present, the dopant may include electron donating or electron withdrawing functional groups or elements.

In embodiments, the second end 14 may be modified to include or be in contact with an electrical conductive material. As used herein, an electrical conductive material may be one or more of aluminum, brass, bronze, copper, gold, graphite, iron, mercury, palladium, platinum, silver, aluminum or steel.

Energy Storage Device

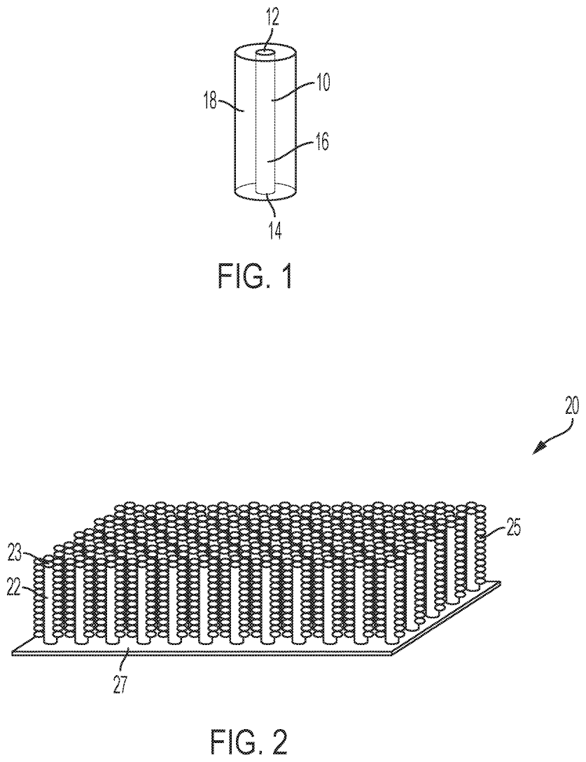

Referring to FIG. 2, in one or more embodiments, a device for energy storage 20 may include a plurality of highly densified CNT rods 22, a plurality of cations 23, and a current collector 25. As described above, each CNT rod may include an insulating surface layer and at least one aligned CNT fiber embedded in the insulating surface layer. Each of the at least one aligned CNT fiber has a first end and a second end opposite the first end, and the first end and the second end are separated by a body. Each of the at least one aligned CNT fiber is composed of a plurality of CNTs. The first end and the second end are free of the insulating surface layer. The first end is in contact with the plurality of cations 23. The second end is in contact with the current collector. In embodiments, as shown in FIG. 2, the highly densified CNT rods 22 may be intercalated with towers 27 of cation producing compounds, such as lithium ion producing compounds.

In embodiments, the at least one aligned CNT fiber may be embedded in the insulating surface layer. The insulating surface may be made from epoxy containing resin, solvent- and water-borne polyurethane, polysiloxane, polyphosphazene, synthetic organic polymers that have rigidity for cutting, and mixtures of two more of these. When the at least one aligned CNT fiber 10 is embedded in the insulating surface layer 18, the entire assembly may be referred to as a “carbon nanotube rod” or a “CNT rod.”

As used in this context, “at least one” means that any number of aligned CNT fibers may be embedded in the insulating surface layer. For instance, from 1 to 1000 aligned CNT fibers may be embedded in the insulating surface layer. That is, from 1 to 6, from 1 to 12, from 1 to 24, from 1 to 254, from 1 to 10, from 1 to 9, from 1 to 8, from 1 to 7, from 1 to 6, from 1 to 5, from 1 to 4, from 1 to 3, from 1 to 2, from 2 to 254, from 3 to 254, from 4 to 254, from 5 to 254, from 6 to 254, from 7 to 254, from 8 to 254, from 9 to 254, from 10 to 254, from 2 to 1000, from 3 to 1000, from 4 to 1000, from 5 to 1000, from 6 to 1000, from 7 to 1000, from 8 to 1000, from 9 to 1000, or even from 10 to 1000 aligned CNT fibers may be embedded in the insulating surface layer. It should be understood that the number of aligned CNT fibers embedded in the insulating surface layer may be from any of the lower bounds of such number described herein to any of the upper bounds of such number described herein.

In embodiments, the at least one aligned CNT fiber may be densified. Without intending to be bound by any particular theory, it is believed that densification of the CNT fiber may help to limit the amount of porosity in the CNT fiber. As used herein, the term “porosity” refers to the relative amount of open space within the CNT fiber 10, with “high porosity” referring to a large amount of open space within the CNT fiber and “low porosity” referring to a small amount of open space within the CNT fiber. In either case, the porosity may be sufficient to allow solvent to penetrate and disperse within the CNTs. Additionally, it is believed that porosity may affect the electrochemical response of the electrodes or sensors formed from the CNT fiber 10. Without intending to be bound by any particular theory, it is believed that porosity will be evident for fibers densified for 30 minutes in acetone (i.e., partial densification), allowing electrolyte migration leading to thin film behavior. These fibers typically lead to transition directly from radial diffusion to thin layer effect as the scan rate increases. In porous CNT fiber, peak to peak separation (ΔE) also decreases (as shown in FIG. 12 described in more detail below, where the ΔE value observed is 42 mV for single fiber, rather than 59 mV). Additionally, it is believed that porosity may affect the electrochemical response of the energy storage devices formed from the CNT fiber due to the pores being dimensioned so as to accommodate ions present in the energy storage device. Densification is also believed to improve alignment of the individual CNTs within the CNT fiber and may also increase the conductivity of the CNT fiber.

Densification may be accomplished by exposing the CNT fiber to a non-solvent at a temperature and for a period of time. For instance, the non-solvent may be selected from acetone, a mixture of water and acetone, ethylene glycol, N-methyl-2-pyrrolidone, and a mixture of two or more of these. Voltage can also be applied to densify fiber. Densification may take place, for example, for a time ranging from 18 hours to 54 hours, from 22 hours to 50 hours, from 26 hours to 46 hours, from 30 hours to 42 hours, or even from 34 hours to 38 hours. It should be understood that densification may take place for a time ranging from any lower bound for such time described herein to any upper bound of such time described herein. Further, densification may take place, for example, at a temperature ranging from 0° C. to 100° C., from 5° C. to 95° C., from 10° C. to 90° C., from 15° C. to 85° C., from 20° C. to 80° C., from 25° C. to 75° C., from 30° C. to 70° C., from 35° C. to 65° C., from 40° C. to 60° C., or even from 45° C. to 55° C. It should be understood that densification may take place at a temperature ranging from any lower bound for such temperature described herein to any upper bound of such temperature described herein.

In embodiments, each of the at least one aligned CNT fiber is composed of a plurality of CNTs. It is believed that the total number of CNTs in a single CNT fiber, in embodiments, may be one million or more, such as up to 10²³ CNTs. Of course, the total number of CNTs in a single CNT fiber may vary based on the dimensions of the CNT fiber and the like.

The CNTs in a single CNT fiber may have an average length of from 20 μm to 60 μm, from 21 μm to 59 μm, from 22 μm to 58 μm, from 23 μm to 57 μm, from 24 μm to 56 μm, from 25 μm to 55 μm, from 26 μm to 54 μm, from 27 μm to 53 μm, from 28 μm to 52 μm, from 29 μm to 51 μm, from 30 μm to 50 μm, from 31 μm to 49 μm, from 32 μm to 48 μm, from 33 μm to 47 μm, from 34 μm to 46 μm, from 35 μm to 45 μm, from 36 μm to 44 μm, from 37 μm to 43 μm, from 38 μm to 42 μm, or even from 39 μm to 41 μm. It should be understood that the CNTs may have an average length ranging from any lower bound for such length described herein to any upper bound for such length described herein. Without intending to be bound by any particular theory, it is believed that this length may allow for a continuous electron path from the first end of the CNT fiber to the second end of the CNT fiber, which in turn, may allow for fast electron transfer while the CNT fiber is in operation.

Referring again to FIG. 2, in embodiments, the first end and the second end may be free of the insulating surface layer. In embodiments, the insulating surface layer may be absent. In such embodiments, the insulating surface layer may be included initially to aid processing, but then removed prior to operation of the energy storage device. Without intending to be bound by any particular theory, it is believed that assembling the CNT rod such that the first end and the second end are free of the insulating surface layer allows for access to the first end and the second end to connection points for any desired electronic leads.

In embodiments, the first end may be modified to include one or more hydrogen atoms, one or more carbon atoms, a chemical functional group, a polymer, a nanoparticle, an enzyme, an aptamer, an antibody, a dopant, or a combination of two or more of these. In embodiments, the first end may be in contact with, or may be modified to include, a plurality of cations. For instance, the plurality of cations may include lithium ions.

In embodiments, when a chemical functional group is present, such a chemical functional group may include carboxylic, hydroxyl, amine, silane, thiol, epoxy, oxygen, or a combination of two or more of these.

In embodiments, when a polymer is present, such a polymer may include a conducting polymer, an ion-exchange polymer, a redox polymer, a silyl-modified polymer, a hydrogel polymer, or a combination of two or more of these.

In embodiments, when a nanoparticle is present, such a nanoparticle may include a gold nanoparticle, a silver nanoparticle, a carbon nanoparticle, a palladium nanoparticle, a copper nanoparticle, a platinum nanoparticle, a nickel nanoparticle, or a combination of two or more of these. As used in this context, a “combination of two or more of these” refers to (1) particles comprising two or more metals, e.g. a gold/palladium particle; (2) a mixture of pure particles, e.g. a mixture of gold particles and palladium particles; and/or a combination of these, e.g. a mixture of gold particles, palladium particles, and gold/palladium particles.

In embodiments, when an enzyme is present, such an enzyme may include horseradish peroxidase, glucose oxidase, nicotinamide adenine dinucleotide, organophosphorus hydrolase, cholesterol oxidase, alkaline phosphatase, or a combination of two or more of these.

In embodiments, when an aptamer is present, the aptamer may comprise either chains of oligonucleotides or chains of peptides. For instance, the aptamer may comprise from 20 oligonucleotide to 60 oligonucleotides, from 20 oligonucleotide to 55 oligonucleotides, from 20 oligonucleotide to 50 oligonucleotides, from 20 oligonucleotide to 45 oligonucleotides, from 20 oligonucleotide to 40 oligonucleotides, from 20 oligonucleotide to 35 oligonucleotides, from 20 oligonucleotide to 30 oligonucleotides, from 25 oligonucleotide to 60 oligonucleotides, from 30 oligonucleotide to 60 oligonucleotides, from 35 oligonucleotide to 60 oligonucleotides, from 40 oligonucleotide to 60 oligonucleotides, from 45 oligonucleotide to 60 oligonucleotides, or even from 50 oligonucleotide to 60 oligonucleotides. It should be understood that the aptamer may comprise a number of oligonucleotides ranging from any lower bound for such number described herein to any upper bound for such number described herein. For an aptamer comprising peptides, the aptamer may comprise from 1 to 20 peptides, from 1 to 19 peptides, from 1 to 18 peptides, from 1 to 17 peptides, from 1 to 16 peptides, from 1 to 15 peptides, from 1 to 14 peptides, from 1 to 13 peptides, from 1 to 12 peptides, from 1 to 11 peptides, from 1 to 10 peptides, from 1 to 9 peptides, from 1 to 8 peptides, from 1 to 7 peptides, from 1 to 6 peptides, from 1 to 5 peptides, from 1 to 4 peptides, from 1 to 3 peptides, from 1 to 2 peptides, from 2 to 20 peptides, from 3 to 20 peptides, from 4 to 20 peptides from 5 to 20 peptides, from 6 to 20 peptides, from 7 to 20 peptides, from 8 to 20 peptides, from 9 to 20 peptides, from 10 to 20 peptides, from 11 to 20 peptides, from 12 to 20 peptides, from 13 to 20 peptides, from 14 to 20 peptides, from 15 to 20 peptides, from 16 to 20 peptides, from 17 to 20 peptides, from 18 to 20 peptides, from 19 to 20 peptides.

In embodiments, when an antibody is present, the antibody may be specific to any antigen. Antigens may originate from any pathogen, including pathogenic bacteria and viruses.

In embodiments, when a dopant is present, the dopant may include electron donating or electron withdrawing functional groups or elements.

In embodiments, the second end may be modified to include an electrical conductive material. As used herein, an electrical conductive material may be one or more of aluminum, brass, bronze, copper, gold, graphite, iron, mercury, palladium, platinum, silver, or steel. In embodiments, the second end may be in contact with the current collector 25. In embodiments, the current collector 25 may comprise one or more of aluminum, brass, bronze, copper, gold, graphite, iron, mercury, palladium, platinum, silver, or steel.

Assembly of CNT Rods and CNT Films

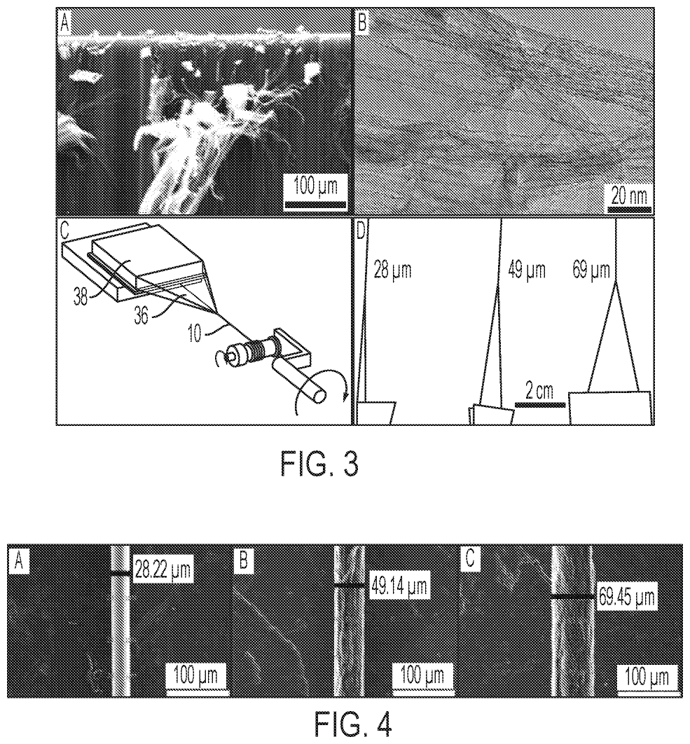

CNT-rods may be fabricated by first synthesizing a vertically aligned (VA) CNT forest that has the ability to assemble into ribbons and fibers. That is the VA CNT forest is composed of drawable or spinnable CNTs. Fibers of different diameters may be assembled from the drawable CNT arrays having various widths. FIG. 4, panel A, shows the field emission scanning electron microscopy (FE-SEM) images of VA CNTs grown by chemical vapor deposition (CVD) on a silicon dioxide (SiO₂) substrate. Typical heights of VA CNTs are from 150 μm and 450 μm. FIG. 3, panel B, shows the transition electron microscopy (TEM) image of representative individual CNTs extracted from the VA CNT forest.

FIG. 3, panel C, is a schematic of the fiber fabrication process from VA CNT forest arrays using the dry spinning method. A CNT film 36 may drawn from a VA CNT forest 38 and simultaneously spun into a CNT fiber 10. By changing the size (i.e. width) of the VA CNT forest 38, CNT fibers 10 of different diameters may be prepared. For instance, exemplary CNT fibers 10 have been prepared to have diameters of 28 μm, 49 μm, and 69 μm, as shown in FIG. 3, panel D, which is an optical image of CNT fiber fabrication from different width VA CNT forests. The fiber diameters were confirmed by FE-SEM, as shown in FIG. 4, panels A, B, and C. The as-spun CNT fibers may then be densified to produce a non-porous electrode material. An exemplary densification may be conducted by placing the CNT fiber in acetone for from 1 hour to 96 hours, for instance for 96 hours, at 30° C.

Any number of the densified CNT fibers may be embedded in the insulating surface layer. The CNT fibers may be separately placed in a mold containing the ingredients of the insulating surface lawyer. The insulating surface layer may then be cured by applying heat. For instance, the insulating surface layer may be heated for a time at a temperature sufficient for curing the insulating surface layer. The insulating surface layer may be cured for a time ranging from 12 hours to 36 hours, from 13 hours to 35 hours, from 14 hours to 34 hours, from 15 hours to 33 hours, from 16 hours to 32 hours, from 17 hours to 31 hours, from 18 hours to 30 hours, from 19 hours to 29 hours, from 20 hours to 28 hours, from 21 hours to 27 hours, from 22 hours to 26 hours, or even from 23 hours to 25 hours. It should be understood that the insulating surface layer may be cured for a time ranging from any lower bound for such time described herein to any upper bound of such time described herein. Further, the insulating surface layer may be cured, for example, at a temperature ranging from 50° C. to 100° C., from 55° C. to 95° C., from 60° C. to 90° C., from 65° C. to 85° C., or even from 70° C. to 80° C. It should be understood that the insulating surface layer may be cured at a temperature ranging from any lower bound for such temperature described herein to any upper bound of such temperature described herein.

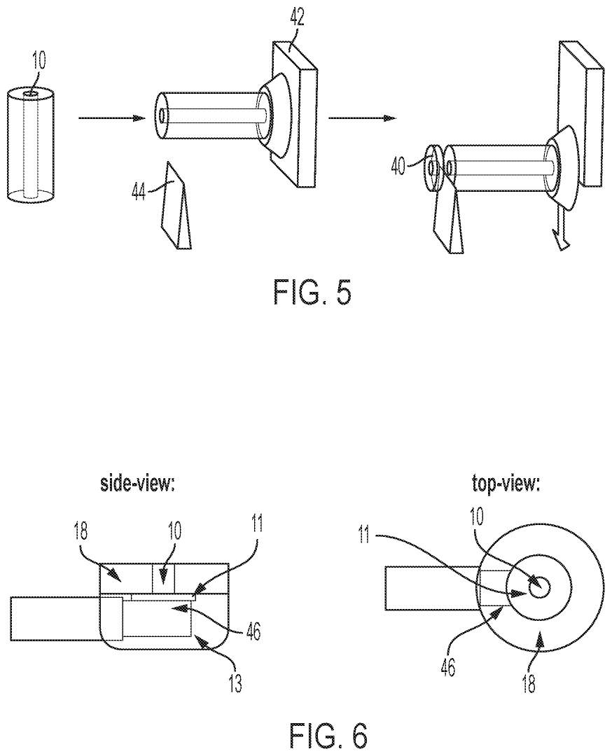

The CNT rods thus produced may be used to produce CNT films. A schematic of an exemplary method of producing such CNT films 40 is shown in FIG. 5. A CNT rod containing a CNT fiber 10 may be placed in a microtome sample holder 42. CNT films 40 may be sliced from the CNT rod by moving the CNT rod in the microtome sample holder 42 toward a blade 44 that is held stationary. Alternatively, blade 44 may be moveable, and the sample holder 42, and thus the CNT rod, may be held stationary. As shown in FIG. 6, an electrical conductive material 46 may be attached to the CNT films 40 thus produced, using a conductive paste 11, for example. In embodiments, the attached CNT films 40 and electrical conductive material 46 may be encapsulated in a protective material 13.

Method of Analyzing an Analyte

In one or more embodiments, a method for analyzing an analyte in a sample includes contacting the sample with a sensor comprising an electrode as described above, applying an electrical potential to the electrode, and measuring the electrical current in the sample as a result of the applied electrical potential. The sample may include very small concentrations of the analyte. For instance, the sample may include 100 ppm by weight or less of the analyte.

Exemplary analyte and sample pairings include heavy metals in an aqueous solution or suspension; pesticides in one or more of soil, an aqueous solution, an aqueous suspension, or air; or one or more gas phase molecules in air. As used herein, an “aqueous solution or aqueous suspension” includes water from natural sources (e.g., lake water, river water, sea water, spring water), drinking water, tap water, reverse osmosis treated water, deionized water, soil, blood, sweat, urine, and a mixture of two or more of these.

Defects at the cross section of the surface of CNT rod electrodes are believed to be oxygen functional groups. These oxygen functional groups may be converted to other functional groups (such as amino-, thiol-, and biomolecules i.e. aptamers and enzymes) for in vitro and in vivo biosensing. In embodiments, the analyte may be a biomolecule, such as dopamine, serotonin, monoamines, epinephrine, nor-epinephrine, histamine, phenethylamine, N-methylphenethyl-amine, tyramine, octopamine, synephrine, N-methyltryptamine, tryptamine and the like, amino acids (such as glutamate, aspartate, D-serine, γ-aminobutyric acid (GABA), glycine and the like), gasotransmitters (such as nitric oxide (NO), carbon monoxide (CO), hydrogen sulfide (H₂S) and the like), peptides, oxytocin, somatostatin, substance P, cocaine and amphetamine regulated transcript, opioid peptides, and the like, nitrogenous base, purines, pyrimidine, ribonucleoside, deoxyribonucleoside, acetylcholine (ACh), anandamide, ascorbic acid, xanthine, hypoxanthine, urea, world anti-doping agency prohibited drugs, β-blocker drugs, amoxicillin, domperidone, paracetamol, melatonin, antibiotics, and proton-pump inhibitor drugs (such as omeprazole, pantoprazole, and the like). In embodiments, the analyte may comprise one or more neurotransmitters, antidoping drugs, nucleic acids, beta blocker drugs, peptides, steroids and hormones.

In embodiments, the electrode may include a plurality of electroactive sites spaced such that the analyte maintains non-overlapping hemispherical diffusion profiles to each electroactive site so that each site functions as an independent electrode when analyzing a sample.

In embodiments, the end of the electrode exposed to the sample has a high density of aggregated open-ended CNTs that constitute chemically active sites employed for heavy metal detection in an aqueous solution or suspension. These electrochemically active open-ended CNTs serve as nanoscale electrodes and can act as the working, counter and reference electrodes in a three-electrode sensor system, or as the working and reference electrodes in a two-electrode sensor system. Without intending to be bound by any particular theory, it is believed that by aggregating nanoscale individual CNTs into cylindrical rod-like structures with micrometer dimensions, fractal characteristics (micro- and nano-features) are produced that appear to benefit the high sensitivity of these sensors.

In embodiments, CNT rods may be employed in an electronic nose, or e-nose. The e-nose provides potential benefits to various commercial industries related to environment, food, cosmetics, biomedical, pharmaceuticals, and agriculture. E-nose is widely used for pollution measurement, medical diagnosis, environment monitoring and food quality control. Electrodes described herein may be applied for the sensing of gas molecules and volatile organic compounds (VOCs) using amperometric and voltammetric analysis. In these techniques, by applying a potential on electrode, the gaseous molecule adsorbed on the cross-section of CNT rod surface will be oxidized or reduced and generate a measurable current. These electrodes are inexpensive and mass deployable in polluted areas, near pipeline junctions, to detect the gas leakage. Along with this, sensors described herein may be capable of detecting the gases exhaled by human lungs, which in term of medical potential, can be used to identify the content in each exhale to identify the symptoms of diseases for real time monitoring.

The electrode described herein may include a number of CNT rods embedded in polymer film and may operate as a sensor film to detect gases that are commonly responsible for pollution or that are indicative of dysfunction of biological systems of organisms. For instance, gases found in breath samples are carbon monoxide (CO), carbon dioxide (CO2), sulfur dioxide (SO₂), N₂, O₂, H₂, NO, NO₂, and methane (CH₄). Sensing VOCs in breath may also be interesting avenues of research for diagnosis of various diseases. Although there is no specific compound or gas in the breath, typically, which can identify a disease, abnormal level of gases can help in diagnosis. VOCs are mostly linked to respiratory diseases and perhaps, but lung and breast cancer are also heavily studied research areas for sensing the VOCs in the breath using e-nose.

Aspects

In a first aspect, either alone or in combination with any other aspect, an electrode includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material.

In a second aspect, either alone or in combination with any other aspect, the first end comprises one or more hydrogen atoms, one or more carbon atoms, a chemical functional group, a polymer, a nanoparticle, an enzyme, an aptamer, an antibody, a dopant, or a combination of two or more of these.

In a third aspect, either alone or in combination with any other aspect, the at least one aligned carbon nanotube fiber is densified.

In a fourth aspect, either alone or in combination with any other aspect, the first end comprises a chemical functional group selected from the group consisting of carboxylic, hydroxyl, amine, silane, thiol, epoxy, oxygen, and a combination of two or more of these.

In a fifth aspect, either alone or in combination with any other aspect, the first end comprises a polymer selected from the group consisting of a conducting polymer, an ion-exchange polymer, a redox polymer, a silyl-modified polymer, a hydrogel polymer, and a combination of two or more of these.

In a sixth aspect, either alone or in combination with any other aspect, the first end comprises a nanoparticle selected from the group consisting of a gold nanoparticle, a silver nanoparticle, a carbon nanoparticle, a palladium nanoparticle, a copper nanoparticle, a platinum nanoparticle, a nickel nanoparticle, and a combination of two or more of these.

In a seventh aspect, either alone or in combination with the sixth aspect, the nanoparticle is functionalized with a polymer or a chemical functional group selected from the group consisting of carboxylic, hydroxyl, thiol, amine, oxygen, and a combination of two or more of these.

In an eighth aspect, either alone or in combination with any other aspect, the first end comprises an enzyme selected from the group consisting of horseradish peroxidase, glucose oxidase, nicotinamide adenine dinucleotide, organophosphorus hydrolase, cholesterol oxidase, alkaline phosphatase, and a combination of two or more of these.

In a ninth aspect, either alone or in combination with any other aspect, the at least one aligned carbon nanotube fiber comprises an electrode or microelectrode array.

In a tenth aspect, either alone or in combination with any other aspect, the at least one aligned carbon nanotube fiber comprises a single electrode or microelectrode.

In an eleventh aspect, either alone or in combination with any other aspect, a method for analyzing an analyte in a sample includes contacting the sample with a sensor comprising an electrode of any of the above aspects, applying an electrical potential to the electrode, and measuring the electrical current in the sample as a result of the applied electrical potential. The sample comprises 100 ppm by weight or less of the analyte.

In a twelfth aspect, either alone or in combination with any other aspect, the analyte comprises heavy metals and the sample comprises an aqueous solution or suspension.

In a thirteenth aspect, either alone or in combination with any other aspect, the sample comprises an aqueous solution or suspension selected from the group consisting of lake water, river water, sea water, spring water, drinking water, tap water, reverse osmosis treated water, deionized water, soil, blood, sweat, urine, and a mixture of two or more of these.

In a fourteenth aspect, either alone or in combination with any other aspect, the analyte comprises one or more pesticides and the sample comprises one or more of soil, an aqueous solution, an aqueous suspension, or air.

In a fifteenth aspect, either alone or in combination with any other aspect, the analyte comprises one or more neurotransmitters, antidoping drugs, nucleic acids, beta blocker drugs, peptides, steroids and hormones.

In a sixteenth aspect, either alone or in combination with any other aspect, the electrode comprises a plurality of electroactive sites, each of the plurality of electroactive sites spaced such that the analyte maintains a hemispherical diffusion to the electrode.

In a seventeenth aspect, either alone or in combination with any other aspect, the analyte comprises a gas phase molecule and the sample comprises air.

In an eighteenth aspect, either alone or in combination with any other aspect, the electrode is affixed to a surface and the sample contacts the surface such that the electrode is capable of providing continuous, real time monitoring of the analyte in the sample.

In a nineteenth aspect, either alone or in combination with any other aspect, a device for energy storage includes a plurality of highly densified carbon nanotube rods. The highly densified carbon nanotube rods includes an insulating surface layer and at least one aligned carbon nanotube fiber embedded in the insulating surface layer. Each of the at least one aligned carbon nanotube fiber has a first end and a second end opposite the first end, and the first end and the second end are separated by a body. Each of the at least one aligned carbon nanotube fiber is composed of a plurality of carbon nanotubes. The first end and the second end are free of the insulating surface layer. The second end is in contact with an electrical conductive material.

In a twentieth aspect, either alone or in combination with any other aspect, an assay device includes the electrode of any of the above aspects, a counter electrode, and a reference electrode.

In a twenty-first aspect, either alone or in combination with any other aspect, an assay device includes the electrode of any of the above aspects and a counter/reference combination electrode.

### EXAMPLES

The embodiments described herein will be further clarified by the following examples.

### Example 1—CNT Rods

CNT fibers were produced by the fiber fabrication process from vertically aligned CNT forest arrays using the dry spinning method, as described above. The CNT fibers were then embedded in the insulating surface layer material, thereby producing a CNT rod, which was composed of 43% by volume EMBed-812, 36% dodecenylsuccinic anhydride, and 18% N-methylol acrylamide, together with 3% benzyl dimethylamine. To embed the CNT fibers in the insulating surface layer, the insulating surface layer materials were placed in capsule-shaped 2 ml microcentrifuge plastic tubes, and then the CNT fibers were placed inside the microcentrifuge tube at the desired location.

To prepare the CNT rod embedded polymer film, the capsule prepared as above was positioned in a microtome, with the fiber length perpendicular to the cutting blade. Slicing of the polymer capsule was carried out at an identical thickness of 40 μm for each film, and cross-sections of the CNT rods were exposed at both sides of the sliced film. With the aid of an optical microscope, silver paste was applied on one end of CNT rod cross-section (reverse side) of the 40 μm thick polymer film and in order to make an electrical connection, a conductive metal wire was attached with silver paste. After drying, the silver paste was encapsulated with epoxy resin for electrical insulation. The front side of the polymer film was used to investigate the electrochemical behavior at the open ends of the CNT rod electrodes.

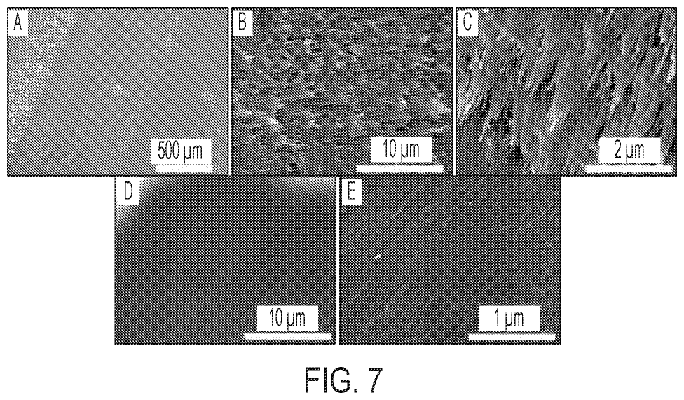

The active surface area of CNT rod electrodes was imaged using SEM, the resulting cross-sections are shown in FIG. 7, panels A, B, C, D, and E. FIG. 7, panel A, shows the top view of three CNT-rods cross-sections, where the distance between each CNT rod cross-sections is 10 times greater than their diameter. FIG. 7, panels B and C, show a cross-section of non-densified CNT rods at different magnifications, in which the porosity within the CNT rod is observed. Cross-sections of densely packed CNTs can be seen in FIG. 7, panel D, which is a representative cross-section of CNT rod electrodes. FIG. 7, panel E provides the same cross-section at higher magnification.

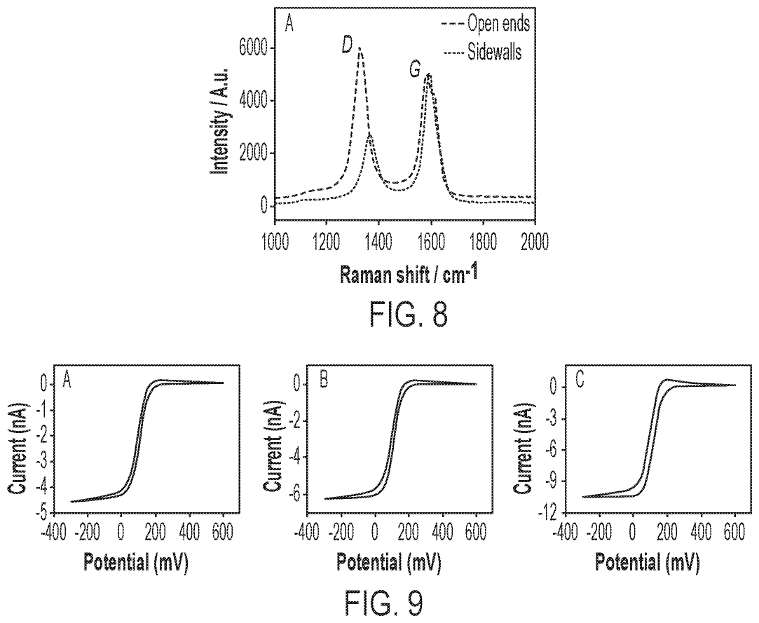

Raman spectra were recorded using a 633 nm laser with spot size 3 μm and 10% attenuation at different positions on the CNT rod electrode cross-section and side walls of CNT fibers. In FIG. 8, the Raman spectra of the sidewall and cross-section of CNT fiber shows two characteristic peaks. The position and intensity of the D band (ID sp³ carbon) and G band (IG sp² carbon) for sidewalls were observed at 1360 cm⁻¹ and 1592 cm⁻¹. For the cross-section, the position of the D and G bands shifted to 1325 cm⁻¹ and 1585 cm⁻¹, respectively, which is typical during functionalization. The ratio of ID/IG intensity increased for the cross-section as compared to sidewalls of the CNTs. This is most likely a consequence of cutting CNT rods using the microtome blade, after which the freshly exposed surface may create defects which will react with O₂ or H2O, thereby causing a shift in the peak position and an increase in the relative intensity. This ratio increases from 0.5 for the sidewall to 1.2 for the cross-section of CNT rod.

Most of the electrochemical characterization was performed in a glass capillary electrochemical setup (LB16, Dagan, 1.65 mm outer diameter, 1.10 mm inner diameter), where a capillary was pulled into a thin pipette using a micropipet puller (Model P-87, Sutter). The micropipette was polished flat using a micropipette beveller (BV-10, Sutter) to yield a microcapillary with an inner diameter of 50-70 μm. A silver chloride-coated silver (Ag/AgCl) wire was used as a quasi-reference/counter electrode (QRCE), placed inside the capillary along with the desired solution, e.g. 2 mM K₃[Fe(CN)₆] in 0.1 M KCl. The CNT rod cross-section embedded in a polymer film was used as the working electrode, in a two-electrode electrochemical set up. The microcapillary was positioned over the substrate using a 3-axis micro-manipulator system (Sutter MPC-385, Novato, Calif.), and capillary movement and meniscus landing on the cross section of CNT fiber was regulated using a video camera (PL-B776U, Pixelink) with a 2× magnification lens (44 mm, InfiniStix, Edmund Optics). A potential was applied to the substrate using a Dagan Chem-Clamp low noise potentiostat, and cyclic voltammetry was performed at potential scan rates of 10 mV·s⁻¹. The experiments were performed in a humidity controlled cell environment to avoid evaporation of meniscus of the microcapillary electrochemical method (MCEM).

The polymer film, which consisted of multiple CNT rod electrodes with the cross-section exposed, and a single compartment two electrode cell assembly were used to carry out the electrochemical measurements. CNT rod cross-sections were used as the working electrodes and a Ag/AgCl wire was used as a quasi-reference/counter electrode. The Dagan potentiostat was used to measure currents up to 100 nA and electrochemical experiments at the sidewalls of freely suspended, non-insulated CNT fibers with exposed side walls that are available for electrochemistry were recorded using a voltammetric analyser Epsilon EC-USB (BASi, West Lafayette, USA), which had a greater current range. These sidewall experiments are in contrast to the polymer insulated walls of the CNT rods where the first end is the only region of the CNT fiber available for electrochemistry.

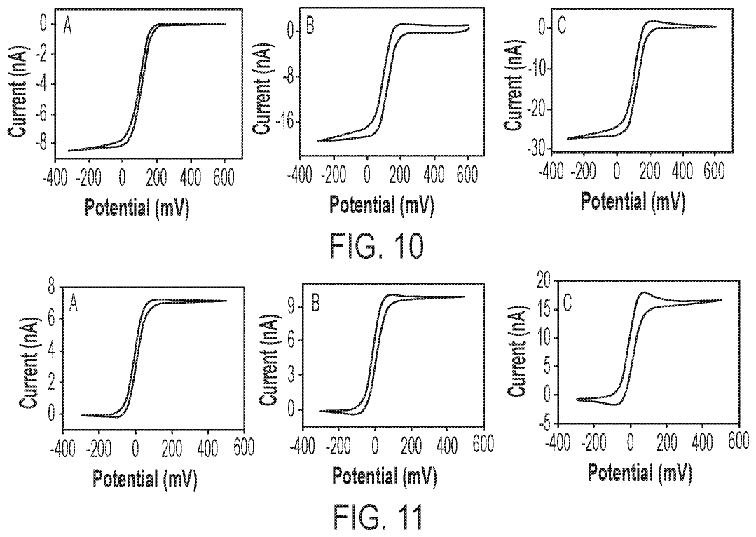

Cyclic voltammograms (CVs) were recorded on CNT rod electrodes with the cross-sections exposed, which had diameters, as measured using SEM, of 28 μm, 49 μm, and 69 μm. These CNT rods were all 40 μm in length (film thickness), with one or three rods of an identical diameter in each film. The three CNT rod cross-sections embedded in the polymer film were identical for each diameter. By using a single compartment two electrode cell assembly, the cyclic voltammetric response was measured in a solution of 2 mM K₃[Fe(CN)₆] in 0.1 M KCl supporting electrolyte at 10 mV·s⁻¹ scan rate, as shown in FIG. 9 for a single fiber and FIG. 10 for three fibers. In FIG. 9, each voltammogram corresponds to the cross section of a single CNT rod, and in FIG. 10, each voltammogram corresponds to three identical CNT rod electrodes with varying diameters of 28 μm, 49 μm, and 69 μm.

It can be seen that CNT rod cross-sections, i.e. open ends, show a sigmoidal steady-state limiting current with a magnitude of several nA, which is characteristic of hemispherical diffusion at ultra microelectrodes. The radial diffusion-controlled limiting plateau current, iₗᵢₘ, at the cross-section of this CNT rod is given by the equation

iₗᵢₘ=4nFDaC

where n refers to the number of electrons transferred per redox event, F is the Faraday constant 96485 C mol⁻¹, D is the diffusion coefficient (7.6×10-6 cm² s⁻¹), C is the bulk concentration of analyte and a is the radius of the CNT rod cross-section electrode. For the largest diameter CNT rod cross-section electrode (FIG. 9, panel C and FIG. 10, panel C; 69 μm diameter), there is a slight deviation from steady-state behavior. With this largest diameter electrode, radial diffusion takes longer to establish and mass transport is likely to have an increased contribution from planar diffusion. Therefore, the larger electrode does not behave as a microelectrode under these conditions, as evidenced by the small oxidation current peak in the CV, meaning the electrode diameter is not comparable to or smaller than the diffusion layer thickness. The electrochemical results at the open ends of the CNT rods show reversible and fast electron transfer, i.e. the difference in the ¼-wave and ¾-wave potential, E_{1/4}-E_{3/4}, for all the sensors are in the range of 59-60 mV.

CVs were also recorded in a solution of 2 mM ferrocenemethanol (FcMeOH) and 0.1 M KNO₃ at 28 μm, 49 μm and 69 μm diameter cross sections of one CNT rod. FIG. 11 shows the typical CVs for the oxidation and reduction of the FcMeOH/FcMeOH+ redox couple, recorded at a 10 mV s⁻¹ scan rate. It can be seen that with increased surface area of cross-section, FcMeOH exhibits adsorption on the CNT rod cross-section. Therefore a larger oxidative peak current was observed in the forward scan in comparison to the reduction peak in the reverse scan of CVs. The peak-to-peak separation (ΔEₚ) for the FcMeOH/FcMeOH+ redox couple was measured at a potential rate of 10 mV s⁻¹ and all found to be 60 mV, which is similar to those measured using K₃[Fe(CN)₆].

To examine the thin layer effect that arises due to gaps in the CNT rod that allow solution to penetrate into the sample, CVs were recorded at the 69 μm diameter cross-section of non-densified CNT rod in 2 mM K₃[Fe(CN)₆] and 0.1 M KCl supporting electrolyte. FIG. 12 shows the voltammograms for the cross section of one (FIG. 12, panel A) and three (FIG. 12, panel B) non-densified CNT rods recorded over at range of scan rates 5-150 mV·s⁻¹. For one and three cross-section electrodes, the peak-to-peak separations (ΔEₚ) were observed to be 50 mV and 42 mV (vs. Ag/AgCl), respectively. It can also be seen that with increasing the scan rate, the redox peak current increased but peak-to-peak separation remained the same. The observed results reflect the fact that these non-densified vertically aligned CNT rod electrodes in the polymer film are potentially “porous,” leading to the thin film behavior. In the diffusional regime, peak current for a reversible electron transfer process follows the Randles-Sevcik equation:

iₚ=0.4463(F³/RT)^{1/2}An^{3/2}D^{1/2}Cv^{1/2}

where iₚ refers to the peak current (in A), F is Faraday's constant (96,485 C/mol), R is the gas constant (8.314 J mol⁻¹ K⁻¹), T is the absolute temperature (298 K), A is the surface area of the electrode (cm²), n=1 electron for K₃[Fe(CN)₆], D is the diffusion coefficient (7.6×10⁻⁶ cm² s⁻¹), C is the concentration of K₃[Fe(CN)₆] in mol cm², and v is the scan rate (V s⁻¹). By calculating the slope of iₚ vs. v^{1/2} in the Randles-Sevcik equation, the active surface areas were found to be 24.2×10⁻⁵ cm² and 161.6×10⁻⁵ cm² for one and three cross-sections of 69 μm diameter non-densified CNT rods, respectively. While the standard surface area for one and three cross-section should be 3.7×10⁻⁵ cm² and 11.1×10⁻⁵ cm², respectively. Thus, it can be concluded that the gap between the CNTs allowed the redox solution to penetrate and, due to change in diffusional regime, non-densified CNT rods lead to several magnitude increments in the active surface area. This effect was named as “thin layer behavior” and is believed to greatly impact the electrochemistry on porous CNT surface.

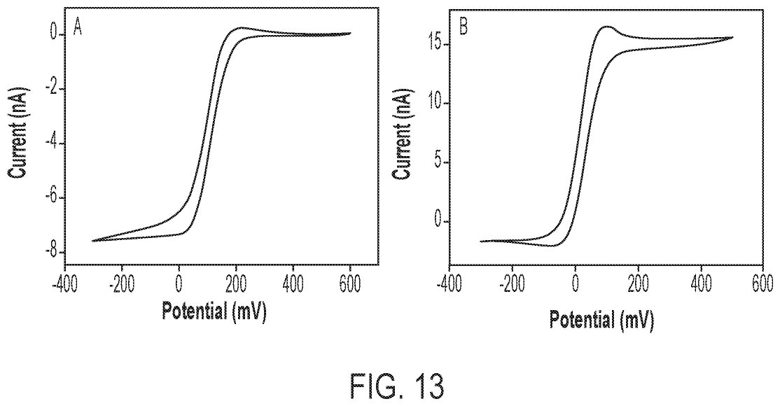

Using the MECM, a 70 μm diameter capillary was filled with the desired redox species and supporting electrolyte. By using a micromanipulator system, the microcapillary was positioned directly above the 69 μm CNT rod cross-section electrode. Once the contact between capillary meniscus and electrode cross-section were made, CVs were recorded. Typical steady-state behavior was observed in CVs for both redox species at a 10 mV s⁻¹ sweep rate (FIG. 13, panels A and B). The voltammetric responses were similar to the previous data, where the difference in the ¼-wave and ¾-wave potential, E_{1/4}-E_{3/4}, was equal to 59 mV, observed for single compartment cell assembly, suggesting fast electron transfer kinetics with ideal reversible behavior at the open end of the CNT rod. As with the previous results, FcMeOH showed strong adsorption at CNT rod cross-section, leading to a large oxidative peak current in the forward scan, as compared to the reverse scan of the CV (FIG. 13, panel B).

The electrochemistry at the sidewalls of CNT fiber was performed using two methods. Firstly, MECM measurements were carried out using a capillary with a 70 μm diameter tip opening, that was positioned at multiple locations along the sidewall of CNT fiber. After the meniscus of the electrolyte solution was brought into contact with the sidewalls of the CNT fiber, CVs were recorded.

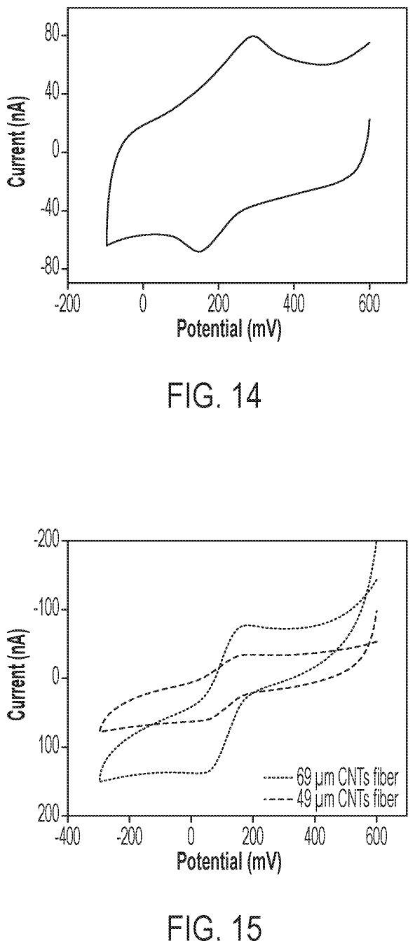

In these MCEM experiments, a microcapillary containing supporting electrolyte and redox analyte was positioned above the CNT fiber sidewall and CVs were recorded at different spots of sidewalls. As shown in FIG. 14, anodic and cathodic peaks at 150 mV and 285 mV (vs. Ag/AgCl) were observed, where peak-to-peak separation (ΔEₚ) was 135 mV, showing irreversible electron transfer behavior at the sidewalls of this CNT fiber.

Secondly, a technique that employed a plastic pipette to confine the solution contact area was used. In this set up, a CNT fiber was inserted through a hole in the wall of a pipette (two holes were made by a needle inserted across the micropipette), which exposed an approximately 0.4 mm to 0.5 mm length of CNT fiber sidewalls to the electrolyte solution. To avoid electrolyte leakage from the pipette, the gaps around the CNT fiber and pipette opening were covered with epoxy resin. For both methods, 2 mM K₃[Fe(CN)₆] redox species and 0.1 M KCl as supporting electrolyte was used to record the CVs at a 10 mV s⁻¹ scan rate.

In the second experiment, i.e. the plastic pipette setup, the electrolyte solution was filled in the pipette and a silver chloride-coated silver wire was placed inside the pipette as a quasi reference-counter electrode (QRCE). CVs were recorded at the different diameters of CNT fibers, and are shown in FIG. 15. From the results, it was observed that with increasing the diameter of CNT fiber, (i.e. 49 μm and 69 μm) the background current increased but oxidation and reduction peaks for all of the CNT fiber sidewalls were found at 165 mV and 40 mV, respectively, suggesting irreversible electrochemical reactions with slower electron transfer rate compares to the open ends of CNTs. The large background or capacitive current can be attributed to the high effective surface area of densely packed carbon nanotubes, which is believed to be working as a bulk carbon material, rather than individual nanotubes.

### Example 2—Sensors for Neurotransmitter Detection: Dopamine, Serotonin, Epinephrine, and Nor-Epinephrine

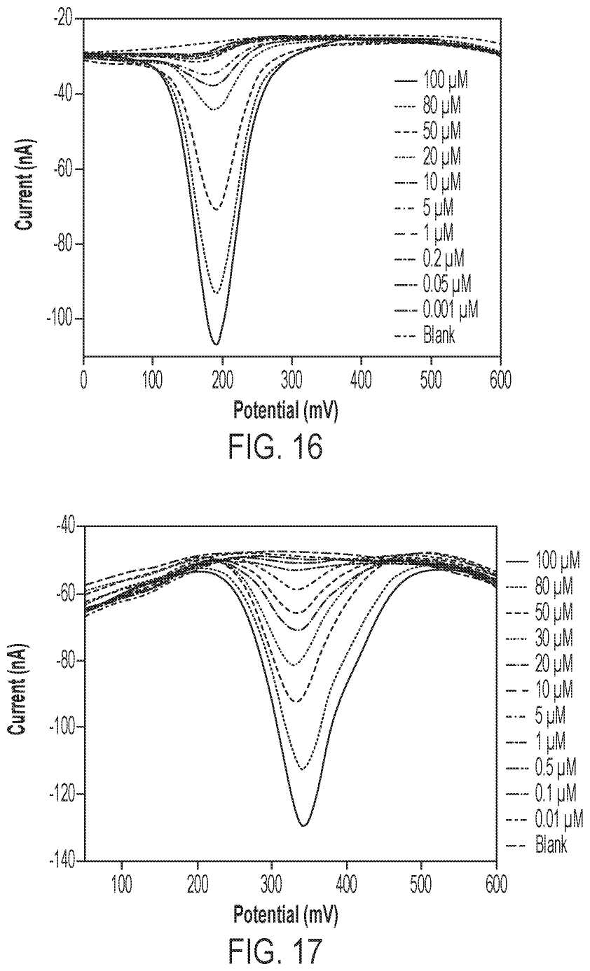

To examine the effect of concentration of dopamine and serotonin on the peak current, square wave voltammograms (SWVs) were recorded at the cross section of six identical CNT rods in phosphate buffer of pH 7.2. The results showed linear increment in the anodic peak current at 190 mV and 340 mV with increasing concentration of dopamine and serotonin, respectively as shown in FIG. 16 and FIG. 17. Concentration investigations were carried out in the range of 1 nM to 100 μM for dopamine and 10 nM to 100 μM for serotonin. The anodic current value dependence on concentration can be expressed by the equations:

iₚ (μA)=0.791[C_{dopamine}(0.001 μM−100 μM)]+3.432 R²=0.998

iₚ (μA)=0.812[Cₛₑᵣₒₜₒₙᵢₙ(0.01 μM−100 μM)]+4.176 R²=0.987

where iₚ is the peak current in nA and C is the concentration of dopamine and serotonin in μM. The limit of detection (LOD) was calculated using 3σ/b, where σ is the standard deviation of “n” number of voltammograms in blank solution and b is the slope of the calibration plot. The LOD for dopamine was found to be 32 pM, and the LOD for serotonin was found to 32.3 pM.

A similar analysis was conducted for epinephrine and nor-epinephrine. The relations produced were as follows.

iₚ (μA)=0.392[Cₑₚᵢₙₑₚₕᵣᵢₙₑ(0.001 μM−100 μM)]+2.253 R²=0.986

iₚ (μA)=0.275[Cₙₒᵣₑₚᵢₙₑₚₕᵣᵢₙₑ(0.001 μM−100 μM)]+0.796 R²=0.992

The LOD for epinephrine was found to be 64 pM, and the LOD for norepinephrine was found to be 91 pM.

Influence of pH

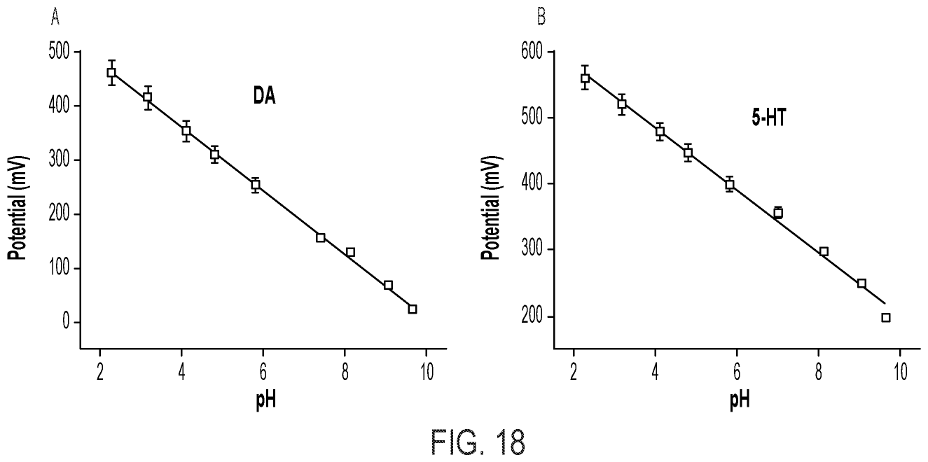

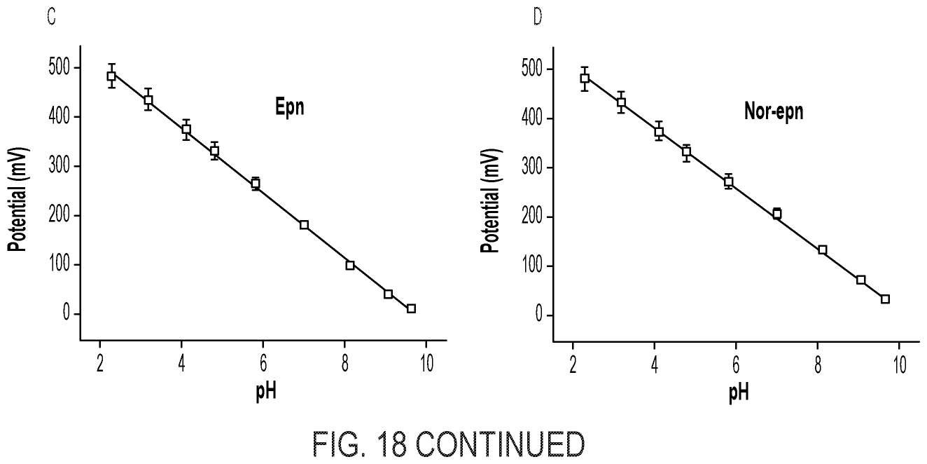

The effect of the pH of the supporting electrolyte on the electro-oxidation of analytes was studied over the pH range of 2.28-9.65 at 10 μM concentration of all individual analytes at the CNT rod microelectrode. The measurements were made three times for all individual analytes at each pH of supporting electrolyte. The oxidation potential of analytes was found to shift toward the less positive potential with an increase in the pH as shown in FIG. 18, panels A, B, C, and D for dopamine, serotonin, epinephrine, and norepinephrine, respectively.

The dEp/pH value for dopamine, serotonin, epinephrine, and norepinephrine was observed close to the Nernst value, i.e., 59 mV/pH, suggesting that an equal number of protons and electrons are participating in the electrochemical oxidation of analytes. The oxidation peak potential of analytes based on the pH of supporting electrolyte versus Ag/AgCl can be expressed by the following equation for dopamine:

Eₚ (pH 2.28−9.65)=−58.625 pH+595.29 mV R²=0.9981;

for serotonin:

Eₚ (pH 2.28−9.65)=−48.129 pH+672.98 mV R²=0.9929;

for epinephrine:

Eₚ (pH 2.28−9.65)=−65.301 pH+640.87 mV R²=0.9986;

and for norepinephrine:

Eₚ (pH 2.28−9.65)=−60.110 pH+621.92 mV R²=0.9988.

Interference Study

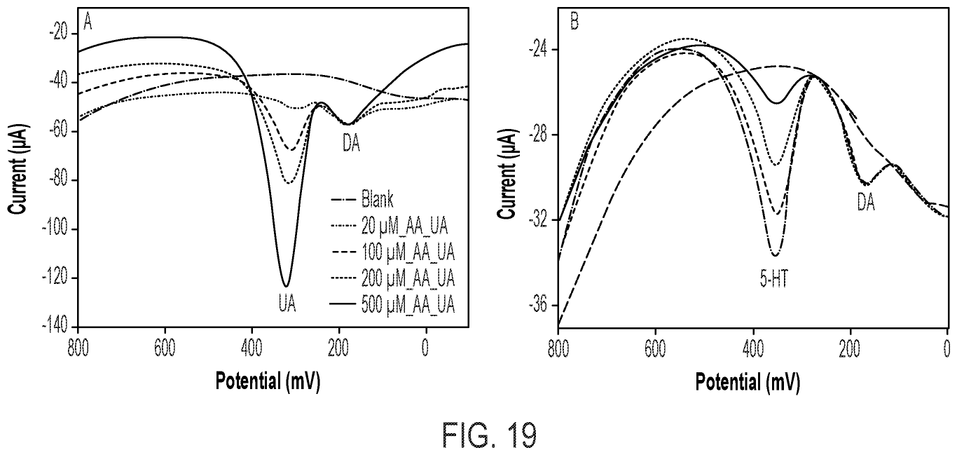

Selective determination of analytes is an important factor for practical applicability due to the presence of various interference molecules in biofluids. Ascorbic acid (AA) and uric acid (UA) are common metabolites present in high concentrations in biological fluids and can interfere with the electrochemical oxidation of DA and affect the quantitative determination. Under optimized conditions, the interference study of DA with AA and UA was performed. FIG. 19, panel A shows the voltammograms recorded for the mixture of AA, dopamine, and UA, where the concentration of dopamine was kept constant (5 μM) and AA and UA concentrations were increased to 500 μM. FIG. 19, panel A, clearly demonstrates the electrochemical oxidation of dopamine was not affected by the concentration of AA and UA, which is 100 times higher than dopamine concentration.

For in vivo studies, AA can interfere with dopamine detection, which is often present in concentrations 100-1000 times more than dopamine is present. CNT fibers may contain negatively charged oxides and carboxyl groups on the surface that may electrostatically repel negatively charged anionic AA and interact with positively charged dopamine. Thus, electrostatically repulsion is believed to inhibit the adsorption and charge transfer of AA at CNT fiber surface. It has also been reported previously that CNT fiber shows supersensitivity toward positively charged dopamine over negatively charged AA and UA. In another experiment, the interference effect of serotonin was investigated at 0.5 μM constant concentration of dopamine. FIG. 19, panel B, presents the observed voltammograms for electrochemical oxidation of 0.5 μM dopamine while increasing the concentration of serotonin (up to 10-fold). Both analytes showed well-separated oxidation peaks, and serotonin peak current was found to increase linearly with increasing concentration without affecting the peak current and peak potential of dopamine.

Real Sample Assay

To evaluate the practical applicability of the developed protocol, dopamine was measured in biological fluids, i.e., urine and serum. Prior to analysis, urine samples were diluted two times with pH 7.4 phosphate buffer solution to reduce the matrix complexity. Now the diluted samples were spiked with a known concentration of standard dopamine solution and SWVs were recorded. The oxidation peaks of dopamine and uric acid were observed at around 180 mV and 330 mV. The peak current for dopamine oxidation increased on spiking dopamine, while the uric acid peak remained constant. The concentration of dopamine was then back-calculated by inserting the observed peak current in the regression equation of the calibration plot and observed data, tabulated in Table 1, showed recovery in the range of 98.80%-102.86% with relative standard deviation (RSD) of ±2.58% (n=3).

TABLE 1

| | Amount added | Amount detected | Recovery |
|---|---|---|---|
| Sample | (μM) | (μM) | (%) |
| Urine | 1 | 0.988 | 98.8 |
| Urine | 5 | 5.143 | 102.86 |
| Urine | 10 | 10.11 | 101.1 |

The proposed sensor also implemented in the evaluation of dopamine in two times buffer diluted human serum sample. The serum sample was spiked with exogenous dopamine, and SWVs were recorded. The observed SWV shows three peaks, i.e., at around 182 mV and 330 mV and a small bump at 692 mV. The analysis report of the serum sample received from the provider shows 205 μM concentration of uric acid, along with 4.55 mM glucose and 305.55 mM protein (albumin and globulin). The peak observed at 330 mV can be associated with uric acid oxidation, while the small bump at 692 mV may be due to xanthine, which is usually present in human serum in a detectable amount. Concentrations of sodium (146 mM), potassium (4 mM), chloride (101 mM), calcium (0.34 mM), and phosphorus (0.20 mM) are also mentioned in analysis reported of the serum sample. However, none of these was found to interfere in the dopamine oxidation peak current. From the observed voltammograms peak currents, dopamine concentration was calculated using regression equation and data have been summarized in Table 2. The dopamine recovery was observed in the range of 97.92-101.60% with RSD of ±3.12%, indicating accuracy and reproducibility of the proposed method. The microelectrode showed clear oxidation peaks for DA oxidation and good recovery results without effect from any interference present in the serum sample. Healthy human serum contains a very low concentration of dopamine near 10⁻¹¹ M or 10⁻¹² M or sometimes rarely reported; therefore it is very hard to detect in such medium. However, since the dopamine concentration is higher in a patient's serum, these microelectrodes may have the potential to successfully detect endogenous dopamine levels. As the deviation in the concentration of catecholamine in the human system leads to various brain disorders, the sensor with pM detection limit can be useful to determine their concentration in patients without any interference from the metabolites present in the human biological fluids.

TABLE 2

| | Amount added | Amount detected | Recovery |
|---|---|---|---|
| Sample | (μM) | (μM) | (%) |
| Serum | 1 | 0.984 | 98.4 |
| Serum | 5 | 4.896 | 97.92 |
| Serum | 10 | 10.16 | 101.6 |

Real Time PC12 Cell Exocytosis Measurements

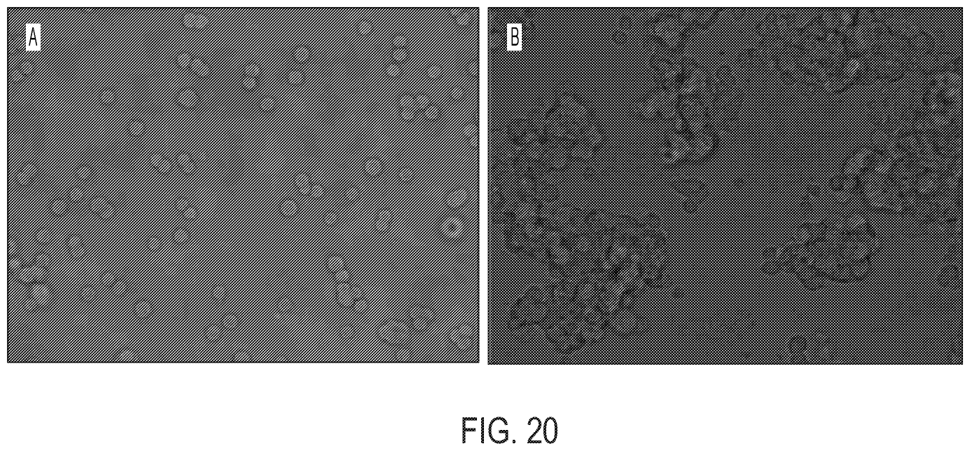

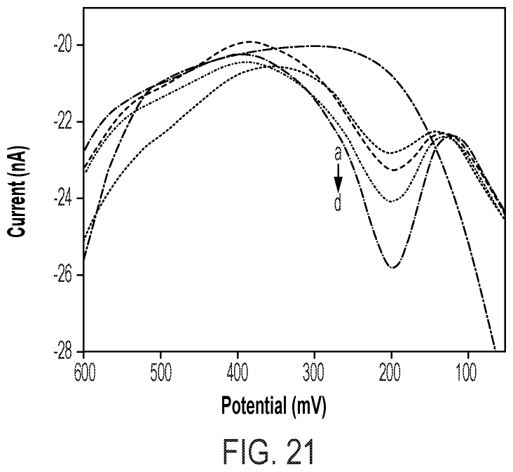

To validate the practical applicability, an ultrasensitive CNT rod sensor was used for the real time dopamine exocytosis measurements of PC12 cells. The PC12 cells were seeded and cultured with a density of 1×10⁷ cells per well/ml (3 ml volume) and 12 samples of cells were prepared in different cell culture plates. FIG. 20, panels A and B, show the microscopic images of PC12 in culture medium at different time intervals. Detection of dopamine release from PC12 cells was performed in cell culture medium. In the first step, the volume of concentrated K⁺ (100 mM) was optimized by gradually increasing the spiked volume from 100 to 600 μl in culture PC12 cells, and SWVs were recorded at each spiked volume of concentrated K⁺ The stimulation of K⁺ is believed to lead to the depolarization of cell membrane, trigger cell exocytosis, and release detectable concentrations of dopamine. It was found that peak current for dopamine release in PC12 cells was increased to 400 μl. FIG. 21 shows the SWVs of K⁺ induced dopamine release from the population of PC12 cells and then further spiked standard dopamine solutions of different concentrations. The peak current was found to increase linearly with increase in the concentration of spiked DA. The measurements were recorded in triplicate (n=3), and the observed correlation equation was

iₚ (μA)=29.04[C_{dopamine}(0.01 μM−0.1 μM)]+3.432 R²=0.99

The standard addition method was applied to measure the concentration of dopamine release from PC12 cells using the above-mentioned voltammetric procedure for the constructed dopamine calibration curve. The recovery values were determined in the range of 98.4%-101.25% for spiked dopamine. The SWVs were also recorded in culture medium (without cells) and then further KCl was also spiked in the detection solution, where no peak current or signal was observed. The concentration of dopamine released after triggering PC12 cells using KCl was calculated using the standard addition method and resulted in 42 nM and sensitivity was calculated to be 22.3×10³ μA μM⁻¹ cm⁻².

### Example 3—Heavy Metal Detection

Lead

A stock solution of 1000 ppb lead was prepared by dissolving the required amount of lead in an amount of de-ionized water. Anodic stripping voltammetry (ASV) was used to detect the lead and cadmium ions in drinking water. Lead ion detection was also performed in pH 4.5 acetate buffer. To record the ASVs, the required amount of stock solution was added in supporting solution over different concentration ranges. Voltammograms were then recorded using the following parameters: deposition potential: −1500 mV; deposition time: 300 s; step potential: 4 mV; frequency: 15 Hz; amplitude: 25 mV; initial potential: −1200 mV; final potential: 0 mV.

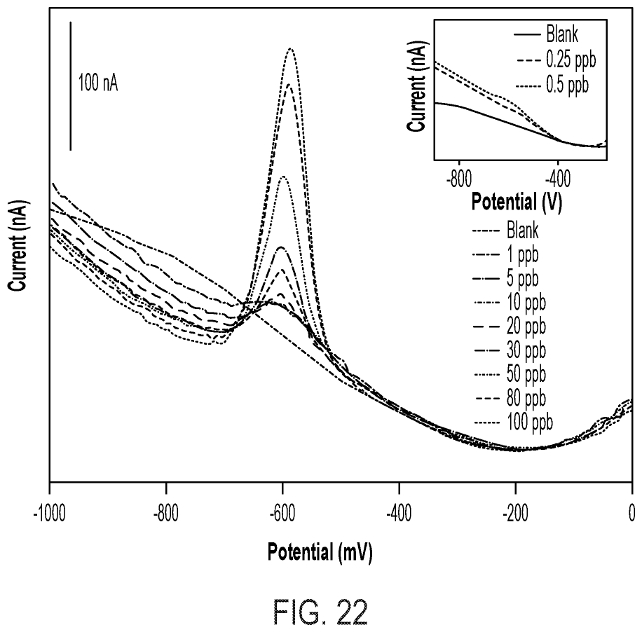

The determination of lead ion concentration in acetate buffer solution of pH 4.5 was performed at six identical CNT rod electrodes encapsulated in an inert polymer film. A well-defined stripping peak for Pb⁺² ions was observed at around 590 mV and an incremental increase in the peak current was observed with increasing concentration of lead ion, as shown in FIG. 22. The effect of lead ion concentration on the peak current can be expressed by the following equation:

iₚ (nA)=1.869[C_{Pb+2}(0.25 ppb−100 ppb)]+7.823 R²=0.994

where iₚ is the peak current in nA and C is the concentration of Pb⁺² metal ion in ppb (parts per billion) in acetate buffer solution. The LOD was calculated using 3σ/b, where σ is the standard deviation of “n” number of voltammograms in the blank solution and b is the slope of calibration plot. The LOD was found to be 2.5 ppt (parts per trillion).

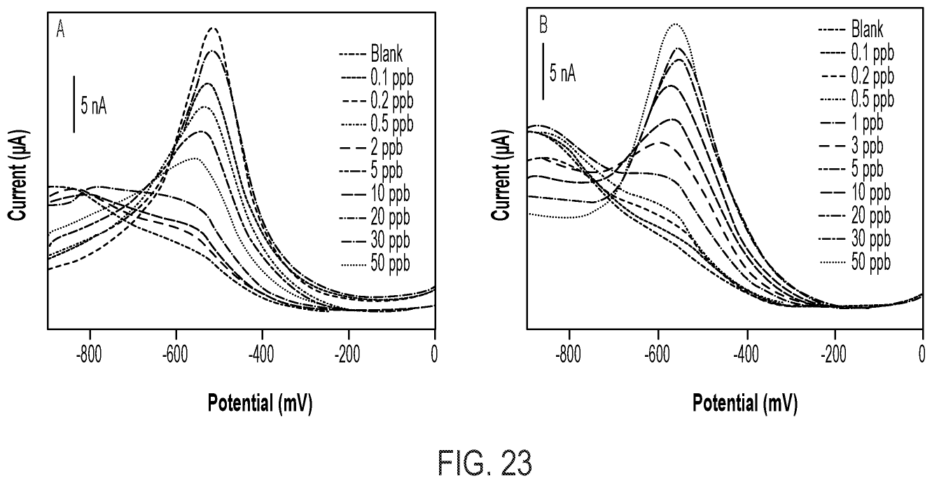

Further lead ion detection was performed in drinking water using six identical CNT rod electrodes. For this investigation, the concentration of lead ion was varied from 0.1 ppb to 100 ppb. The effect of deposition time was examined. The ASV with 300 second deposition time is shown in FIG. 23, panel A. The ASV without deposition time (zero second) is shown in FIG. 23, panel B. From the results, it can be seen that the CNT rod electrode shows good results without the usual deposition time. The calibration graphs appeared to have two linear ranges, at lower concentration and at higher concentration. The dependence of the peak current on lead ion concentration can be expressed by the following equations for the experiments with a deposition time:

iₚ (nA)=2.849[C_{Pb+2}(0.1 ppb−2 ppb)]+2.582 R²=0.999

iₚ (nA)=0.218[C_{Pb+2}(5 ppb−50 ppb)]+8.91 R²=0.991

The dependence of the peak current on lead ion concentration can be expressed by the following equations for the experiments without a deposition time:

iₚ (nA)=1.344[C_{Pb+2}(0.1 ppb−2 ppb)]+0.765 R²=0.987

iₚ (nA)=0.165[C_{Pb+2}(5 ppb−50 ppb)]+9.134 R²=0.995

where iₚ is the peak current in nA and C is the concentration of Pb⁺² metal ion in ppb (parts per billion). The LOD with a deposition time was found to be 1.6 ppt, and the LOD without a deposition time was found to be 3.5 ppt.

Cadmium

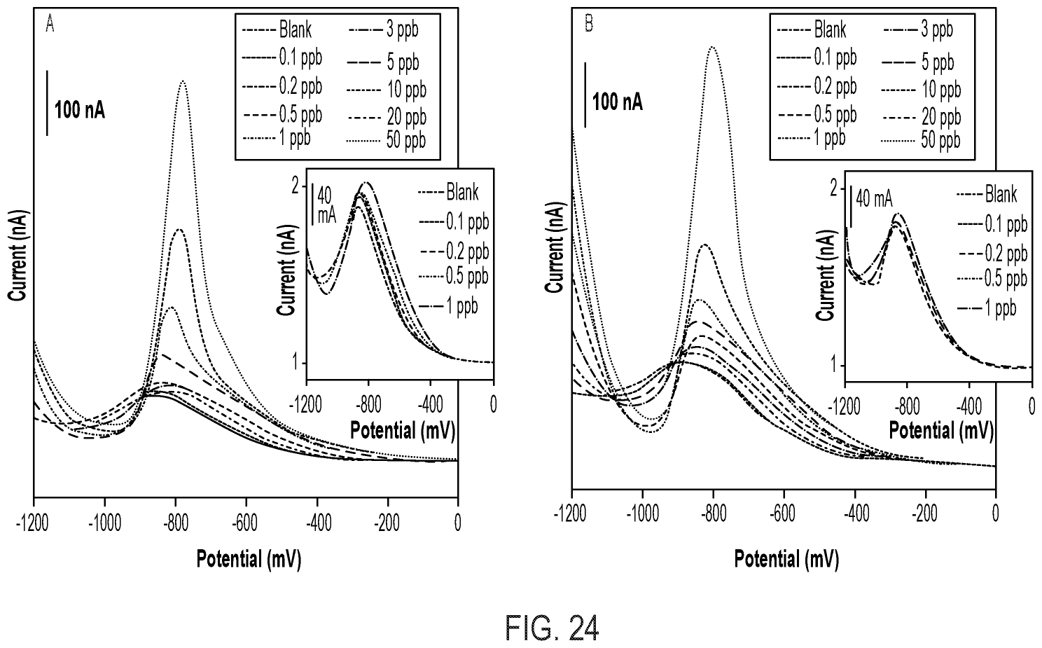

Cadmium detection was investigated in drinking water using the same process as described for lead. To carry out the calibration studies, the concentration of cadmium was varied from 0.1 ppb to 100 ppb. FIG. 24, panel A, shows ASV for a 300 s deposition time, and FIG. 24, panel B, shows ASV without a deposition time. A well-defined stripping peak for Cd⁺² ions was observed at around 780 mV when the deposition time was applied, while a peak at 800 mV was observed without deposition time. An incremental increase in the peak current was observed with increasing concentration of cadmium ions.

The dependence of the peak current on lead ion concentration can be expressed by the following equations for the experiments with a deposition time:

iₚ (nA)=10.44[C_{Cd+2}(0.1 ppb−50 ppb)]+38.58 R²=0.963

The dependence of the peak current on lead ion concentration can be expressed by the following equations for the experiments without a deposition time:

iₚ (nA)=4.99[C_{Pb+2}(0.1 ppb−50 ppb)]+17.06 R²=0.981

where iₚ is the peak current in nA and C is the concentration of Cd⁺² metal ion in ppb (parts per billion). The LOD with a deposition time was found to be 0.45 ppt, and the LOD without a deposition time was found to be 1 ppt.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. An electrode comprising:
an insulating surface layer; and
at least one aligned carbon nanotube fiber embedded in the insulating surface layer, each of the at least one aligned carbon nanotube fiber having a first end and a second end opposite the first end, the first end and the second end separated by a body;
wherein
each of the at least one aligned carbon nanotube fiber is composed of a plurality of carbon nanotubes;
the first end and the second end are free of the insulating surface layer; and
the second end is in contact with an electrical conductive material.

2. The electrode of claim 1, wherein the first end comprises one or more hydrogen atoms, one or more carbon atoms, a chemical functional group, a polymer, a nanoparticle, an enzyme, an aptamer, an antibody, a dopant, or a combination of two or more of these.

3. The electrode of claim 1, wherein the at least one aligned carbon nanotube fiber is densified.

4. The electrode of claim 1, wherein the first end comprises a chemical functional group selected from the group consisting of carboxylic, hydroxyl, amine, silane, thiol, epoxy, oxygen, and a combination of two or more of these.

5. The electrode of claim 1, wherein the first end comprises a polymer selected from the group consisting of a conducting polymer, an ion-Docket exchange polymer, a redox polymer, a silyl-modified polymer, a hydrogel polymer, and a combination of two or more of these.

6. The electrode of claim 1, wherein the first end comprises a nanoparticle selected from the group consisting of a gold nanoparticle, a silver nanoparticle, a carbon nanoparticle, a palladium nanoparticle, a copper nanoparticle, a platinum nanoparticle, a nickel nanoparticle, and a combination of two or more of these.

7. The electrode of claim 6, wherein the nanoparticle is functionalized with a polymer or a chemical functional group selected from the group consisting of carboxylic, hydroxyl, thiol, amine, oxygen, and a combination of two or more of these.

8. The electrode of claim 1, wherein the first end comprises an enzyme selected from the group consisting of horseradish peroxidase, glucose oxidase, nicotinamide adenine dinucleotide, organophosphorus hydrolase, cholesterol oxidase, alkaline phosphatase, and a mixture of two or more of these.

9. The electrode of claim 1, wherein the at least one aligned carbon nanotube fiber comprises an electrode array or microelectrode array.

10. The electrode of claim 1, wherein the at least one aligned carbon nanotube fiber comprises a single electrode or a single microelectrode.

11. A method for analyzing an analyte in a sample, the method comprising
contacting the sample with a sensor comprising the electrode of claim 1;
applying an electrical potential to the electrode; and
measuring the electrical current in the sample as a result of the applied electrical potential;
wherein the sample comprises 100 ppm by weight or less of the analyte.

12. The method of claim 11, wherein the analyte comprises heavy metals and the sample comprises an aqueous solution or suspension.

13. The method of claim 12, wherein the sample comprises an aqueous solution or suspension selected from the group consisting of lake water, river water, sea water, spring water, drinking water, tap water, reverse osmosis treated water, deionized water, soil, blood, sweat, urine, and a mixture of two or more of these.

14. The method of claim 11, wherein the analyte comprises one or more pesticides and the sample comprises one or more of soil, an aqueous solution, an aqueous suspension, or air.

15. The method of claim 11, wherein the analyte comprises one or more neurotransmitters, antidoping drugs, nucleic acids, beta blocker drugs, peptides, steroids and hormones.

16. The method of claim 11, wherein the electrode comprises a plurality of electroactive sites, each of the plurality of electroactive sites spaced such that the analyte maintains a hemispherical diffusion to the electrode.

17. The method of claim 11, wherein the analyte comprises a gas phase molecule and the sample comprises air.

18. The method of claim 11, wherein the electrode is affixed to a surface and the sample contacts the surface such that the electrode is capable of providing continuous, real time monitoring of the analyte in the sample.

19. (canceled)

20. An assay device comprising the electrode of claim 1, a counter electrode, and a reference electrode.

21. An assay device comprising the electrode of claim 1 and a counter/reference combination electrode.
